(19) United States
(12) Patent Application Publication
(10) Pub. No.: US 2019/0365771 A1
(43) Pub. Date: Dec. 5, 2019

(54) APILIMOD COMPOSITIONS AND METHODS FOR USING SAME IN THE TREATMENT OF ALZHEIMER'S DISEASE

(71) Applicant: AI Therapeutics, Inc., Guilford, CT (US)

(72) Inventors: Henri Lichenstein, Guilford, CT (US); Jonathan M. Rothberg, Guilford, CT (US); Chris Conrad, Guilford, CT (US); Marylens Hernandez, Guilford, CT (US)

(21) Appl. No.: 16/340,845

(22) PCT Filed: Oct. 11, 2017
(86) PCT No.: PCT/US17/56147

§ 371 Date: Apr. 10, 2019

Related U.S. Application Data
(60) Provisional application No. 62/407,186, filed on Oct. 12, 2016.

(51) Int. Cl. A61K 31/5377; A61K 9/00; A61P 25/28

(52) U.S. Cl. CPC A61K 31/5377; A61P 25/28; A61K 9/0053

(57) ABSTRACT

The present disclosure relates to methods for treating Alzheimer's disease with apilimod and related compositions and methods.

### CROSS-REFERENCE TO RELATED APPLICATIONS

This application is a national stage entry, filed under 35 U.S.C. § 371, of International Application No. PCT/US2017/056147, filed on Oct. 11, 2017, which claims priority to U.S. Provisional Patent Application No. 62/407,186 filed Oct. 12, 2016, the contents of which are hereby fully incorporated by reference.

### FIELD OF THE DISCLOSURE

The present disclosure relates to compositions comprising apilimod and methods of using same.

### BACKGROUND OF THE DISCLOSURE

Apilimod, also referred to as STA-5326, hereinafter “apilimod”, is recognized as a potent transcriptional inhibitor of IL-12 and IL-23. See e.g., Wada et al. Blood 109 (2007): 1156-1164. IL-12 and IL-23 are inflammatory cytokines normally produced by immune cells, such as B-cells and macrophages, in response to antigenic stimulation. Autoimmune disorders and other disorders characterized by chronic inflammation are characterized in part by inappropriate production of these cytokines. In immune cells, the selective inhibition of IL-12/IL-23 transcription by apilimod was recently shown to be mediated by apilimod's direct binding to phosphatidylinositol-3-phosphate 5-kinase (PIKfyve). See, e.g., Cai et al. Chemistry and Biol. 20 (2013):912-921. PIKfyve plays a role in Toll-like receptor signaling, which is important in innate immunity.

Amyloid precursor protein (APP) is processed by proteases, first by beta secretase (BACE1) and then by gamma secretase to generate peptide fragments, including 40 and 42 amino acid peptides, named Abeta (Ab), e.g., Ab 1-40 and Ab 1-42 respectively. Several familial Alzheimer's disease related mutations and truncated mutants in the APP gene have been described in the investigation of APP processing to Ab in vitro and in vivo. The present disclosure relates to method of reducing Ab formation in a mammal.

### SUMMARY OF THE DISCLOSURE

In one aspect, the present disclosure provides a method for treating Alzheimer's disease in a subject in need thereof, the method comprising administering to the subject a therapeutically effective amount of an apilimod composition of the disclosure, said composition comprising apilimod, or a pharmaceutically acceptable salt, solvate, clathrate, hydrate, polymorph, prodrug, analog or derivative thereof. In embodiments, the apilimod composition comprises apilimod free base or apilimod dimesylate. In embodiments, the method further includes administering at least one additional active agent to the subject. The at least one additional active agent may be a therapeutic agent or a non-therapeutic agent. The at least one additional active agent may be administered in a single dosage form with the apilimod composition, or in a separate dosage form from the apilimod composition. In embodiments, the at least one additional active agent is chosen from cholinesterase inhibitors (Aricept, Exelon, Razadyne), memantine (Namenda), and combinations thereof. In embodiments, the at least one active agent is a non-therapeutic agent selected to ameliorate one or more side effects of the apilimod composition. In embodiments, the non-therapeutic agent is selected from the group consisting of ondanestron, granisetron, dolsetron, and palonosetron. In embodiments, the non-therapeutic agent is selected from the group consisting of pindolol and risperidone. In embodiments, the dosage form of the apilimod composition is an oral dosage form. In another aspect, the dosage form of the apilimod composition is suitable for intravenous administration; administration is by a single injection or by a drip bag.

In embodiments, the subject is a human Alzheimer's disease patient. In embodiments, the human Alzheimer's disease patient in need of treatment with an apilimod composition of the disclosure is on whose Alzheimer's disease is refractory to a standard regimen.

### BRIEF DESCRIPTION OF THE DRAWINGS

FIGS. 1A-1B are bar graphs showing effects of apilimod on the levels of Abeta40 (Aβ40) (FIG. 1A) and Abeta42 (Aβ42) (FIG. 1B) from APP wildtype Hela cells. The abeta (Aβ) concentration is in picogram (10⁻¹² gram) per milliliter (pg/mL).

FIGS. 2A-2B are bar graphs showing effects of apilimod (FIG. 2A) and DAPT (FIG. 2B) on C99 APP truncated mutant (APP C99) mutant cells. In the APP C99 cells C99 fragment encoding the last 99-amino acid of APP 695 mimics the BACE1 cleaved APP at the major Asp+1 site of Aβ to generate C99.

FIG. 3 is a pie chart showing the disease category composition of the Disease Signature Database.

FIG. 4 is a chart showing the top significant diseases identified by the XSum metric as potential indications for Apilimod. Alzheimer's disease was the top indication when diseases were ranked by the drug-disease score.

### DETAILED DESCRIPTION OF THE DISCLOSURE

The present disclosure provides compositions and methods related to the use of apilimod for treating Alzheimer's disease in a subject, preferably a human subject, in need of such treatment. The present disclosure also provides unique biomarkers of apilimod sensitivity. Such biomarkers may find utility in treating Alzheimer's disease by identifying patients whose Alzheimer's disease will be responsive to apilimod therapy. In addition, the present disclosure provides novel therapeutic approaches to Alzheimer's disease treatment based upon combination therapy utilizing apilimod and at least one additional therapeutic agent. The combination therapies described herein exploit the unique cytotoxic activity of apilimod which is shown to provide a synergistic effect when combined with other anti-Alzheimer's disease agents.

As used herein, the term “apilimod” may refer to apilimod itself, or may encompass pharmaceutically acceptable salts, solvates, clathrates, hydrates, polymorphs, metabolites, prodrugs, analogs or derivatives of apilimod, as described below. The structure of apilimod is shown in Formula I:

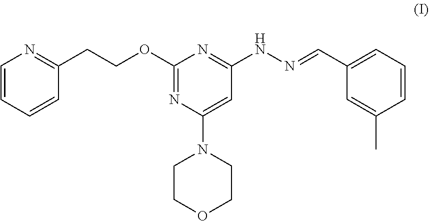 [SMILES: Cc1cccc(/C=N/Nc2cc(N3CCOCC3)nc(OCCc3ccccn3)n2)c1]

The chemical name of apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine), and the CAS number is 541550-19-0.

Apilimod can be prepared, for example, according to the methods described in U.S. Pat. Nos. 7,923,557, and 7,863,270, and WO 2006/128129.

As used herein, the term “pharmaceutically acceptable salt,” is a salt formed from, for example, an acid and a basic group of a compound described herein (e.g., 2-[2-Pyridin-2-yl)-ethoxy]-4-N′-(3-methyl-benzilidene)-hydrazino]-6-(morpholin-4-yl)-pyrimidine). Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (e.g., 1,1′-methylene-bis-(2-hydroxy-3-naphthoate)) salts. In a preferred embodiment, the salt of apilimod comprises methanesulfonate. The term “pharmaceutically acceptable salt” also refers to a salt prepared from a compound described herein (e.g., 2-[2-Pyridin-2-yl)-ethoxy]-4-N′-(3-methyl-benzilidene)-hydrazino]-6-(morpholin-4-yl)-pyrimidine), having an acidic functional group, such as a carboxylic acid functional group, and a pharmaceutically acceptable inorganic or organic base.

Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N, N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound described herein (e.g., 2-[2-Pyridin-2-yl)-ethoxy]-4-N′-(3-methyl-benzilidene)-hydrazino]-6-(morpholin-4-yl)-pyrimidine), having a basic functional group, such as an amino functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, and p-toluenesulfonic acid.

The salts of the compounds described herein (e.g., 2-[2-Pyridin-2-yl)-ethoxy]-4-N′-(3-methyl-benzilidene)-hydrazino]-6-(morpholin-4-yl)-pyrimidine) can be synthesized from the parent compound (e.g., 2-[2-Pyridin-2-yl)-ethoxy]-4-N′-(3-methyl-benzilidene)-hydrazino]-6-(morpholin-4-yl)-pyrimidine) by conventional chemical methods such as methods described in Pharmaceutical Salts: Properties, Selection, and Use, P. Hemrich Stalil (Editor), Camille G. Wermuth (Editor), ISBN: 3-90639-026-8, August 2002. Generally, such salts can be prepared by reacting the parent compound (e.g., 2-[2-Pyridin-2-yl)-ethoxy]-4-N′-(3-methyl-benzilidene)-hydrazino]-6-(morpholin-4-yl)-pyrimidine) with the appropriate acid in water or in an organic solvent, or in a mixture of the two.

One salt form of a compound described herein (e.g., 2-[2-Pyridin-2-yl)-ethoxy]-4-N′-(3-methyl-benzilidene)-hydrazino]-6-(morpholin-4-yl)-pyrimidine) can be converted to the free base and optionally to another salt form by methods well known to the skilled person. For example, the free base can be formed by passing the salt solution through a column containing an amine stationary phase (e.g. a Strata-NH₂ column). Alternatively, a solution of the salt in water can be treated with sodium bicarbonate to decompose the salt and precipitate out the free base. The free base may then be combined with another acid using routine methods.

As used herein, the term “polymorph” means solid crystalline forms of a compound of the present disclosure (e.g., 2-[2-Pyridin-2-yl)-ethoxy]-4-N′-(3-methyl-benzilidene)-hydrazino]-6-(morpholin-4-yl)-pyrimidine) or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of the present disclosure (e.g., 2-[2-Pyridin-2-yl)-ethoxy]-4-N′-(3-methyl-benzilidene)-hydrazino]-6-(morpholin-4-yl)-pyrimidine) or a salt thereof, which further includes a stoichiometric or non-stoichiometric amount of water bound by non-covalent intermolecular forces.

As used herein, the term “clathrate” means a compound of the present disclosure (e.g., 2-[2-Pyridin-2-yl)-ethoxy]-4-N′-(3-methyl-benzilidene)-hydrazino]-6-(morpholin-4-yl)-pyrimidine) or a salt thereof in the form of a crystal lattice that contains spaces (e.g., channels) that have a guest molecule (e.g., a solvent or water) trapped within.

As used herein, the term “prodrug” means a derivative of a compound described herein (e.g., 2-[2-Pyridin-2-yl)-ethoxy]-4-N′-(3-methyl-benzilidene)-hydrazino]-6-(morpholin-4-yl)-pyrimidine) that can hydrolyze, oxidize, or otherwise react under biological conditions (in vitro or in vivo) to provide a compound of the disclosure. Prodrugs may only become active upon such reaction under biological conditions, or they may have activity in their unreacted forms. Examples of prodrugs contemplated in this disclosure include, but are not limited to, analogs or derivatives of a compound described herein (e.g., 2-[2-Pyridin-2-yl)-ethoxy]-4-N′-(3-methyl-benzilidene)-hydrazino]-6-(morpholin-4-yl)-pyrimidine) that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of the formulae disclosed herein that comprise —NO, —NO₂, —ONO, or —ONO₂ moieties. Prodrugs can typically be prepared using well-known methods, such as those described by Burger's Medicinal Chemistry and Drug Discovery (1995) 172-178, 949-982 (Manfred E. Wolff ed., 5th ed).

In addition, some of the compounds suitable for use in the methods of in this disclosure (e.g., 2-[2-Pyridin-2-yl)-ethoxy]-4-N′-(3-methyl-benzilidene)-hydrazino]-6-(morpholin-4-yl)-pyrimidine) have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms. All such isomeric forms of these compounds are expressly included in the present disclosure. The compounds of this disclosure (e.g., 2-[2-Pyridin-2-yl)-ethoxy]-4-N′-(3-methyl-benzilidene)-hydrazino]-6-(morpholin-4-yl)-pyrimidine) can also be represented in multiple tautomeric forms, in such instances, the disclosure expressly includes all tautomeric forms of the compounds described herein (e.g., there may be a rapid equilibrium of multiple structural forms of a compound), the disclosure expressly includes all such reaction products). All such isomeric forms of such compounds are expressly included in the present disclosure. All crystal forms of the compounds described herein (e.g., 2-[2-Pyridin-2-yl)-ethoxy]-4-N′-(3-methyl-benzilidene)-hydrazino]-6-(morpholin-4-yl)-pyrimidine) are expressly included in the present disclosure.

As used herein, the term “solvate” or “pharmaceutically acceptable solvate,” is a solvate formed from the association of one or more solvent molecules to one of the compounds disclosed herein (e.g., 2-[2-Pyridin-2-yl)-ethoxy]-4-N′-(3-methyl-benzilidene)-hydrazino]-6-(morpholin-4-yl)-pyrimidine). The term solvate includes hydrates (e.g., hemi-hydrate, mono-hydrate, dihydrate, trihydrate, tetrahydrate, and the like).

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound. As used herein, the term “derivative” refers to compounds that have a common core structure, and are substituted with various groups as described herein.

In certain embodiments of the disclosure described herein, apilimod, or a pharmaceutically acceptable salt, hydrate, clathrate, or prodrug of apilimod, as described above, may be provided in combination with one or more additional therapeutic agents. In embodiments, apilimod is provided in combination with ibrutinib. In another aspect, apilimod is provided in combination with vemurafenib. In accordance with any of these embodiments, the apilimod, or a pharmaceutically acceptable salt, solvate, clathrate, hydrate, polymorph, metabolite, prodrug, analog or derivative thereof, may be provided in the same dosage form as the one or more additional therapeutic agents, or in a separate dosage form.

### Methods of Treatment

The present disclosure provides methods for the treatment of dementia, including Alzheimer's disease, in a subject in need thereof by administering to the subject a therapeutically effective amount of apilimod, or a pharmaceutically acceptable salt, solvate, clathrate, hydrate, polymorph, metabolite, prodrug, analog or derivative thereof. The present disclosure further provides the use of apilimod, or a pharmaceutically acceptable salt, solvate, clathrate, hydrate, polymorph, metabolite, prodrug, analog or derivative thereof, for the preparation of a medicament useful for the treatment of Alzheimer's disease.

The present disclosure also provides methods comprising combination therapy for the treatment of Alzheimer's disease. As used herein, “combination therapy” or “co-therapy” includes the administration of a compound described herein, e.g., apilimod, or a pharmaceutically acceptable salt, solvate, clathrate, hydrate, polymorph, metabolite, prodrug, analog or derivative thereof, with at least one additional agent, as disclosed herein, as part of a specific treatment regimen intended to provide the beneficial effect from the co-action of these therapeutic compounds. The at least one additional agent may be a therapeutic agent or a non-therapeutic agent. The beneficial effect of the combination includes, but is not limited to, pharmacokinetic or pharmacodynamic co-action resulting from the combination of therapeutic compounds. The beneficial effect of the combination may also relate to the mitigation of a toxicity, side effect, or adverse event associated with another agent in the combination. “Combination therapy” may be, but generally is not, intended to encompass the administration of two or more of these therapeutic compounds as part of separate monotherapy regimens that incidentally and arbitrarily result in the combinations of the present disclosure.

In the context of combination therapy, administration of apilimod, or a pharmaceutically acceptable salt, solvate, clathrate, hydrate, polymorph, metabolite, prodrug, analog or derivative thereof, may be simultaneous with or sequential to the administration of the one or more additional agents. In another aspect, administration of the different components of a combination therapy may be at different frequencies. The one or more additional agents may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a compound of the present disclosure.

The one or more additional agents can be formulated for co-administration with a compound of the present disclosure in a single dosage form, as described in greater detail herein. The one or more additional agents can be administered separately from the dosage form that comprises the compound of the present disclosure. When the additional agent is administered separately from a compound of the present disclosure, it can be by the same or a different route of administration as the compound of the instant disclosure.

Preferably, the administration of a composition comprising a compound of the present disclosure in combination with one or more additional agents provides a synergistic response in the subject having a disorder, disease or condition of the present disclosure. In this context, the term “synergistic” refers to the efficacy of the combination being more effective than the additive effects of either single therapy alone. The synergistic effect of combination therapy according to the disclosure can permit the use of lower dosages and/or less frequent administration of at least one agent in the combination compared to its dose and/or frequency outside of the combination. The synergistic effect can be manifested in the avoidance or reduction of adverse or unwanted side effects associated with the use of either therapy in the combination alone.

“Combination therapy” also embraces the administration of the compounds of the present disclosure in further combination with non-drug therapies (e.g., surgery or radiation treatment). Where the combination therapy further comprises a non-drug treatment, the non-drug treatment may be conducted at any suitable time so long as a beneficial effect from the co-action of the combination of the therapeutic compounds and non-drug treatment is achieved. For example, in appropriate cases, the beneficial effect is still achieved when the non-drug treatment is temporally removed from the administration of the therapeutic compounds, perhaps by days or even weeks.

In embodiments of the methods described herein, apilimod, or a pharmaceutically acceptable salt, solvate, clathrate, hydrate, polymorph, metabolite, prodrug, analog or derivative thereof, may be administered alone or in combination with at least one additional agent in a method for treating Alzheimer's disease. In embodiments, the apilimod, or a pharmaceutically acceptable salt, solvate, clathrate, hydrate, polymorph, metabolite, prodrug, analog or derivative thereof, and the at least one additional agent are administered in a single dosage form. In another aspect, the apilimod and the at least one additional agent are administered in separate dosage forms. In embodiments, the at least one additional agent is a therapeutic agent. In embodiments, the therapeutic agent is indicated for the treatment of Alzheimer's disease, e.g., an anti-Alzheimer's disease agent. In another aspect, the apilimod is administered in combination with at least one additional agent that is not for the treatment of Alzheimer's disease, e.g., a second agent that serves to mitigate a toxicity or adverse event associated with another active agent being administered in the combination therapy, e.g., apilimod, or a pharmaceutically acceptable salt, solvate, clathrate, hydrate, polymorph, metabolite, prodrug, analog or derivative thereof.

In embodiments, the at least one additional agent is an agent which mitigates one or more side effects of apilimod selected from any of nausea, vomiting, headache, dizziness, lightheadedness, drowsiness and stress. In one aspect of this embodiment, the additional agent is an antagonist of a serotonin receptors, also known as 5-hydroxytryptamine receptors or 5-HT receptors. In one aspect, the additional agent is an antagonist of a 5-HT₃ or 5-HT₁ₐ receptor. In one aspect, the agent is selected from the group consisting of ondansetron, granisetron, dolasetron and palonosetron. In another aspect, the agent is selected from the group consisting of pindolol and risperidone.

In embodiments, the at least one additional agent is an anti-Alzheimer's disease agent selected from a cholinesterase inhibitor (e.g., Aricept, Exelon, Razadyne) and memantine (Namenda).

In embodiments, the at least one additional agent is directed towards targeted therapy, wherein the treatment targets the Alzheimer's disease's specific genes, proteins, or the tissue environment that contributes to Alzheimer's disease progression. This type of treatment blocks the progression of Alzheimer's disease cells while limiting damage to healthy cells.

The term “therapeutically effective amount” refers to an amount of apilimod, or a pharmaceutically acceptable salt, solvate, clathrate, hydrate, polymorph, metabolite, prodrug, analog or derivative thereof, sufficient to treat, ameliorate a symptom of, reduce the severity of, or reduce the duration of the disease, disorder or condition, or enhance or improve the therapeutic effect of another therapy, or to prevent an identified disease, disorder or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration.

An effective amount of apilimod can be administered once daily, from two to five times daily, up to two times or up to three times daily, or up to eight times daily. In embodiments, the apilimod is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

An effective amount of a compound, such as apilimod or a pharmaceutically acceptable salt, solvate, clathrate, hydrate, polymorph, metabolite, prodrug, analog or derivative thereof, can range from about 0.001 mg/kg to about 1000 mg/kg, more preferably 0.01 mg/kg to about 100 mg/kg, more preferably 0.1 mg/kg to about 10 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents. See, e.g., U.S. Pat. No. 7,863,270, incorporated herein by reference.

In more specific aspects, a compound of the disclosure (e.g., apilimod or a pharmaceutically acceptable salt, solvate, clathrate, hydrate, polymorph, metabolite, prodrug, analog or derivative thereof) is administered at a dosage regimen of 30-300 mg/day (e.g., 30, 35, 40, 45, 50, 55, 60, 65, 70, 75, 80, 85, 90, 95, 100, 125, 150, 175, 200, 225, 250, 275, or 300 mg/day) for at least 1 week (e.g., 1, 2, 3, 4, 5, 6, 7, 8, 9, 10, 11, 12, 36, 48, or more weeks). Preferably, a compound of the disclosure is administered at a dosage regimen of 100-300 mg/day for 4 or 16 weeks. Alternatively or subsequently, a compound of the disclosure is administered at a dosage regimen of 100 mg twice a day for 8 weeks, or optionally, for 52 weeks.

As used herein, a “subject in need thereof” is a subject having a disease, disorder or condition, or a subject having an increased risk of developing a disease, disorder or condition relative to the population at large. In a preferred aspect, the subject in need thereof is a subject having Alzheimer's disease or having an increased risk of developing Alzheimer's disease relative to the population at large. The subject in need thereof can be one that is “non-responsive” or “refractory” to a currently available therapy for the disease or disorder. In this context, the terms “non-responsive” and “refractory” refer to the subject's response to therapy as not clinically adequate to relieve one or more symptoms associated with the disease or disorder.

A “subject” includes a mammal. The mammal can be e.g., any mammal, e.g., a human, primate, vertebrate, bird, mouse, rat, fowl, dog, cat, cow, horse, goat, camel, sheep or a pig. Preferably, the mammal is a human. The terms “subject” and “patient” are used interchangeably herein.

The present disclosure provides a monotherapy for the treatment of a disease, disorder or condition as described herein. As used herein, “monotherapy” refers to the administration of a single active or therapeutic compound to a subject in need thereof. Preferably, monotherapy will involve administration of a therapeutically effective amount of an active compound. For example, monotherapy with a compound of the disclosure, or a pharmaceutically acceptable salt, solvate, clathrate, hydrate, polymorph, metabolite, prodrug, analog or derivative thereof, can be administered in a therapeutically effective amount to a subject in need of treatment. Monotherapy may be contrasted with combination therapy, in which a combination of multiple active compounds is administered, preferably with each component of the combination present in a therapeutically effective amount. In one aspect, monotherapy with a compound of the disclosure, or a pharmaceutically acceptable salt, solvate, clathrate, hydrate, polymorph, metabolite, prodrug, analog or derivative thereof, is more effective than combination therapy in inducing a desired biological effect.

As used herein, “treatment”, “treating” or “treat” describes the management and care of a patient for the purpose of combating a disease, condition, or disorder and includes the administration of a compound of the disclosure, or a pharmaceutically acceptable salt, solvate, clathrate, hydrate, polymorph, metabolite, prodrug, analog or derivative thereof, to alleviate the symptoms or complications of a disease, condition or disorder, or to eliminate the disease, condition or disorder.

As used herein, “prevention”, “preventing” or “prevent” describes reducing or eliminating the onset of the symptoms or complications of the disease, condition or disorder and includes the administration of a compound of the disclosure, or a pharmaceutically acceptable salt, solvate, clathrate, hydrate, polymorph, metabolite, prodrug, analog or derivative thereof, to reduce the onset, development or recurrence of symptoms of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of a compound of the disclosure leads to the elimination of a sign or symptom, however, elimination is not required. Effective dosages are expected to decrease the severity of a sign or symptom.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

Treating a disorder, disease or condition according to the methods described herein can result in a decrease in Alzheimer's disease progression rate. Preferably, after treatment, Alzheimer's disease progression rate is reduced by at least 5% relative to number prior to treatment; more preferably, Alzheimer's disease progression rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Alzheimer's disease progression rate may be measured by any reproducible means of measurement.

As used herein, the term “selectively” means tending to occur at a higher frequency in one population than in another population. The compared populations can be cell populations. Preferably, a compound of the present disclosure, or a pharmaceutically acceptable salt, solvate, clathrate, hydrate, polymorph, metabolite, prodrug, analog or derivative thereof, acts selectively on a hyper-proliferating cells but not on a normal cell. A compound of the present disclosure, or a pharmaceutically acceptable salt, solvate, clathrate, hydrate, polymorph, metabolite, prodrug, analog or derivative thereof, acts selectively to modulate one molecular target (e.g., Amyloid precursor protein) but does not significantly modulate another molecular target (e.g., Amyloid precursor protein). The disclosure also provides a method for selectively inhibiting the activity of an enzyme, such as a beta secretase (BACE-1) or gamma secretagse. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B.

### Pharmaceutical Compositions and Formulations

The present disclosure provides pharmaceutical compositions comprising an amount of apilimod, or a pharmaceutically acceptable salt, solvate, clathrate, hydrate, polymorph, metabolite, prodrug, analog or derivative thereof, in combination with at least one pharmaceutically acceptable excipient or carrier, wherein the amount is effective for the treatment of a disease or disorder. In embodiments, the disease or disorder is selected from dementia and Alzheimer's disease.

In embodiments, the apilimod, or a pharmaceutically acceptable salt, solvate, clathrate, hydrate, polymorph, metabolite, prodrug, analog or derivative thereof, is combined with at least one additional agent in a single dosage form. In embodiments, the pharmaceutical composition further comprises an antioxidant.

A “pharmaceutical composition” is a formulation containing the compounds described herein in a pharmaceutically acceptable form suitable for administration to a subject. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). An effective amount of a pharmaceutical composition is that which provides an objectively identifiable improvement as noted by the clinician or other qualified observer. For example, alleviating a symptom of a disorder, disease or condition. As used herein, the term “dosage effective manner” refers to amount of a pharmaceutical composition to produce the desired biological effect in a subject or cell.

For example, the dosage unit form can comprise 1 nanogram to 2 milligrams, or 0.1 milligrams to 2 grams; or from 10 milligrams to 1 gram, or from 50 milligrams to 500 milligrams or from 1 microgram to 20 milligrams; or from 1 microgram to 10 milligrams; or from 0.1 milligrams to 2 milligrams.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the disclosure may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present disclosure with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present disclosure may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present disclosure together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

The tablet can be a coated tablet. The coating can be a protective film coating (e.g. a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

A pharmaceutical composition can be in the form of a hard or soft gelatin capsule. In accordance with this formulation, the compound of the present disclosure may be in a solid, semi-solid, or liquid form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present disclosure as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present disclosure can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value.

Among the surfactants for use in the compositions of the disclosure are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present disclosure also provides packaging and kits comprising pharmaceutical compositions for use in the methods of the present disclosure. The kit can comprise one or more containers selected from the group consisting of a bottle, a vial, an ampoule, a blister pack, and a syringe. The kit can further include one or more of instructions for use in treating and/or preventing a disease, condition or disorder of the present disclosure, one or more syringes, one or more applicators, or a sterile solution suitable for reconstituting a pharmaceutical composition of the present disclosure.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### Example 1

The Amyloid precursor protein (APP) can be processed by proteases, first by beta secretase (BACE1) then followed by gamma secretase to generate peptide fragments including sizes of 40 and 42 amino acids named abeta (Ab), e.g. Ab 1-40 and Ab 1-42. Several familial Alzheimer disease-related mutations and truncated mutants in the APP gene have been described in the investigation of APP processing to Ab in vitro and in vivo. The data presented here demonstrate that there is a dose-dependent decrease in Ab with apilimod treatment in an in vitro model system. Briefly, two constructs were used to generate Ab:(1) APP Swedish/Indiana double mutant (APPSw-I) in the 695 amino acid APP construct combines the Swedish mutant APP K670N, M671L (Mullan M et al, A pathogenic mutation for probable Alzheimer's disease in the APP gene at the N-terminus of beta-amyloid. Nat Genet., 1992 August; 1(5):345-7) and the Indiana mutation APP V717F (Suzuki N et al, An increased percentage of long amyloid beta protein secreted by familial amyloid beta protein precursor (beta APP717) mutants. Science, 1994 May 27; 264(5163): 1336-40); and (2) C99 APP truncated mutant (C99) fragment encoding the last 99-aa of APP 695, this construct mimics the BACE1 cleaved APP at the major Asp+1 site of Aβ to generate C99.

Hela cells were transiently transfected with the two constructs, either APPSw-I or C99 and 24 hours after transfection (to allow sufficient time for Ab production) the cells were then treated for two days with apilimod (LAM-002) at the following doses: 10000 nanomolar (nM), 1000 nM, 100 nM, 1 nM or DMSO. In parallel, a Gamma Secretase inhibitor, DAPT (N-[(3,5-Difluorophenyl)acetyl]-L-alanyl-2-phenyl]glycine-1,1-dimethylethylester) was used as a positive control at concentrations including 1000 nm, 100 nM, 10 nM or 1 nM.

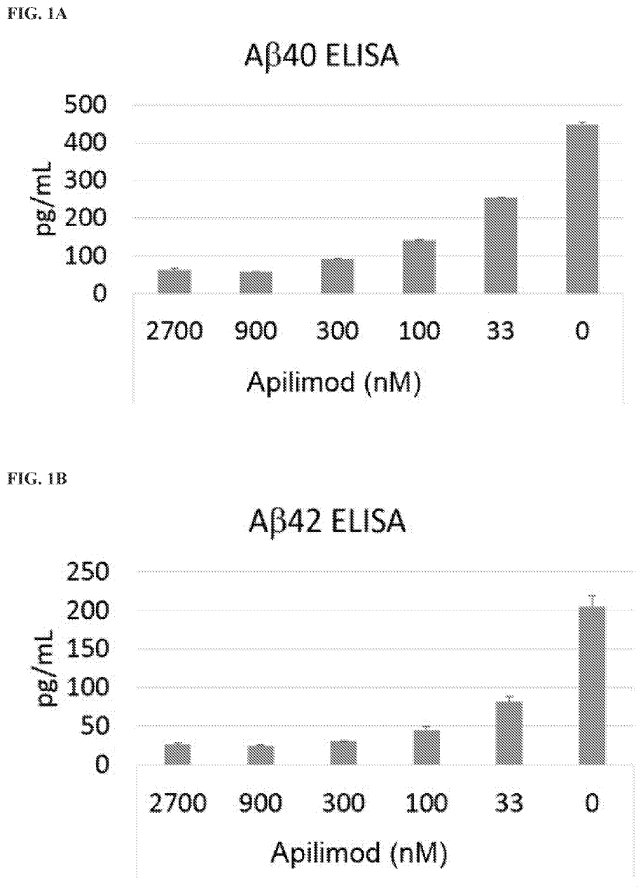

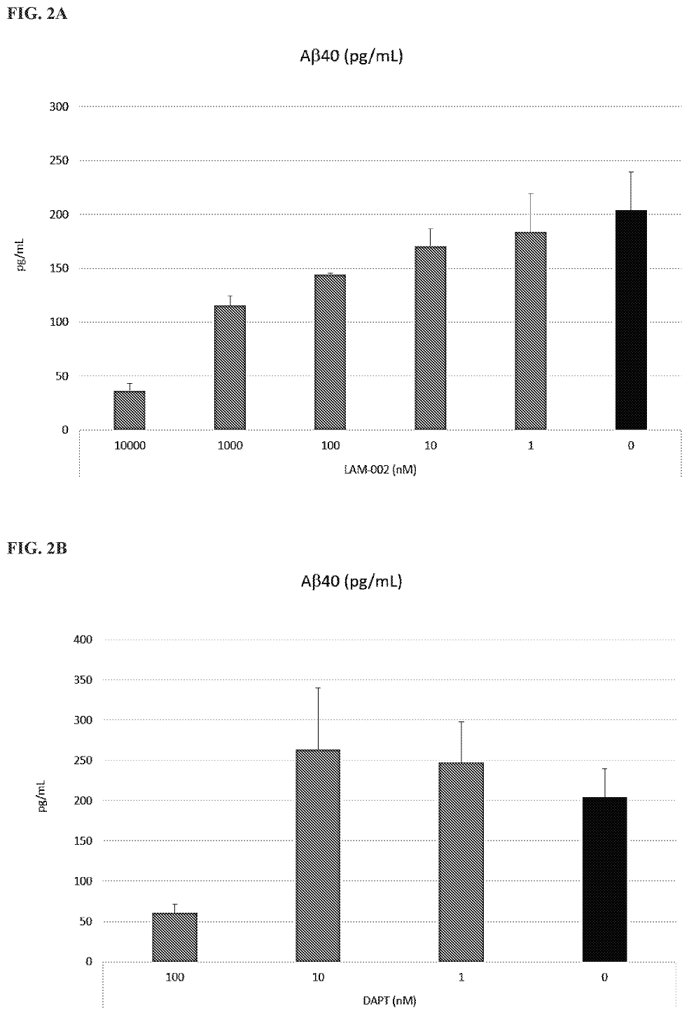

Cell culture supernatants were collected and treated with the protease inhibitor AEBSF (4-(2-Aminoethyl) benzenesulfonyl fluoride hydrochloride). The supernatants were then assayed in the Ab 40 ELISA assay and the data analysis was carried out according to manufacturer's protocols. (SensoLyte Anti-Human β Amyloid (1-40) Quantitative ELISA, Catalog # AS-55551 Anaspec), the results are shown in FIGS. 1 and 2.

FIG. 1 shows the data from an experiment in which cells were transfected with the APPSw-I double mutant construct followed by treatment with apilimod (LAM-002) (left panel) or the positive control, DAPT (right panel). Ab concentration is shown in picogram (10-12 gram) per milliliter (pg/mL).

FIG. 2 shows the data from an experiment in which cells were transfected with the APP C99 mutant construct followed by treatment with apilimod (LAM-002) (left panel) or the positive control, DAPT (right panel).

Both experiments demonstrate a dose dependent decrease of Ab with apilimod treatment.

### Example 2: Computational Approach Identified Alzheimer's Disease as Potential Indication for Apilimod

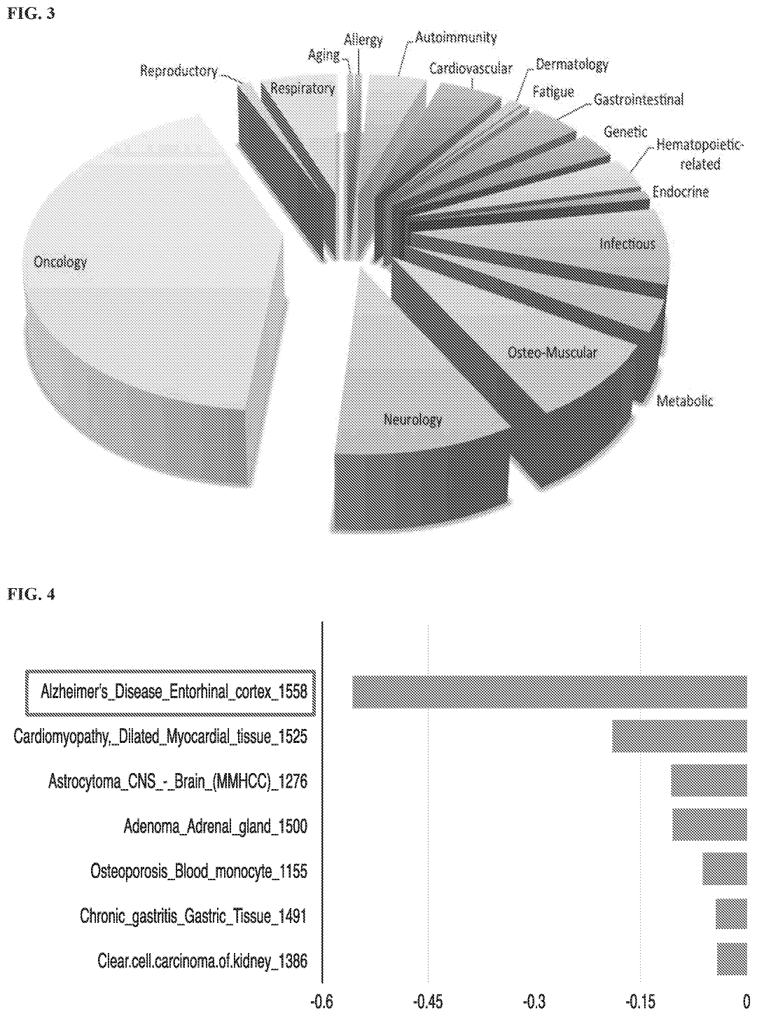

A computational drug repurposing approach was used to identify novel indications for Apilimod. The core algorithm of the analysis compared the gene expression profile induced by Apilimod in multiple cell lines, at different concentrations and time points, to the gene expression signature of multiple diseases. To conduct this comparison, a disease database composed of 210 disease expression signatures was created, that contained genes significantly changed between control/normal and patient samples in different tissue types. The database contained multiple expression signatures per disease and multiple disease categories (FIG. 3).

Apilimod gene expression profiles were generated in house. Seven different cell lines were profiled at different concentrations and time points (Table 1, below). These cell lines were selected because they were previously reported to capture a broad spectrum of the drug perturbation space, and have been successfully used in other drug repurposing projects (Lamb J et al. The Connectivity Map: using gene-expression signatures to connect small molecules, genes, and disease. Science. 2006 Sep. 29; 313(5795):1929-35, 2006; Dudley J T et al. Computational repositioning of the anticonvulsant topiramate for inflammatory bowel disease. Sci Transl Med. 2011 Aug. 17; 3(96); Jahchan N S et al. A drug repositioning approach identifies tricyclic antidepressants as inhibitors of small cell lung cancer and other neuroendocrine tumors. Cancer Discov. 2013 December; 3(12):1364-77).

TABLE 1
Apilimod expression profiles generated in house
with seven different cancer cell lines

| Drug | Cell Line | Dosage (nM) | Duration (hr) |
|---|---|---|---|
| LAM-002 | A549 | 60 | 6 |
| LAM-002 | A549 | 60 | 12 |
| LAM-002 | A549 | 60 | 24 |
| LAM-002 | A549 | 300 | 12 |
| LAM-002 | A673 | 60 | 6 |
| LAM-002 | A673 | 300 | 6 |
| LAM-002 | AGS | 60 | 6 |
| LAM-002 | AGS | 60 | 12 |
| LAM-002 | AGS | 300 | 12 |
| LAM-002 | HepG2 | 300 | 12 |
| LAM-002 | HT29 | 60 | 6 |
| LAM-002 | HT29 | 60 | 12 |
| LAM-002 | HT29 | 300 | 6 |
| LAM-002 | HT29 | 300 | 12 |
| LAM-002 | MCF7 | 60 | 6 |
| LAM-002 | MCF7 | 60 | 12 |
| LAM-002 | MCF7 | 60 | 24 |
| LAM-002 | MCF7 | 300 | 12 |
| LAM-002 | MCF7 | 300 | 24 |
| LAM-002 | VCAP | 60 | 6 |

The disease expression signature database was generated by comparing the expression profile between control/normal and disease samples. These profiles were extracted and manually curated from the NCBI Gene Expression Omnibus (GEO). The differential gene expression between disease and control samples was calculated in R using the RankProd library of the Bioconductor software (https://bioconductor.org). Only genes with a false discovery rate (FDR) lower than 0.05 were considered statistically significant and were included in the disease signature.

Briefly, each disease signature was queried against the rank-ordered drug expression profiles to quantitatively measure the similarity between both profiles, and a score was calculated separately for the up-regulated and down-regulated gene sets. This analysis proposed that if up-regulated disease genes localize at the bottom (down-regulated) of the drug expression profile and the down-regulated disease genes localize at the top (up-regulated) of the drug expression profile, then the drug-disease pair is consider a good match.

Finally, a drug-disease score (dds) was calculated that measures the similarity of the drug and disease expression profiles, and only when the comparison rendered a significant score was the disease considered a potential indication option for Apilimod. Three different metrics were used to compare the drug and disease expression profiles: Enrichment Score (ES), Extreme Sum (XSum) and Extreme Cosine (X Cos) (Cheng J, et al. Systematic evaluation of connectivity map for disease indications. Genome Med. 2014 Dec. 2; 6(12):540).

After calculating the drug-disease scores for each disease profile (FIG. 4), the results of each metric were combined, and diseases were ranked according to the percentage of significant profiles. The ranking was based on the rationale that diseases represented by a higher number of significant profiles, were more likely true indications for the drug.

Results indicated that Alzheimer's disease was within the top five predicted indications for Apilimod (Table 2, below), suggesting that Apilimod could be a treatment option for this disease.

TABLE 2
Top ranked diseases according to the number of
significant profiles across the three metrics

| Disease | % of Significant Profiles (N) |
|---|---|
| Cardiomyopathy | 75 (10) |
| Alzheimer's Disease | 67 (30) |
| Bacterial Infection/Septic Shock | 67 (7) |
| Crohn's Disease | 67 (3) |
| Non-Hodgkin Lymphoma | 60 (8) |

1. A method for treating dementia in a subject in need thereof, the method comprising administering to the subject a pharmaceutical composition comprising apilimod in an amount effective to inhibit the processing of amyloid precursor protein (APP) into abeta (Ab) peptides in a cell of the subject.

2. The method of claim 1, wherein the cell is a neural cell.

3. The method of claim 1, wherein the pharmaceutical composition is an oral dosage form.

4. The method of claim 1, further comprising administering to the subject at least one additional agent.

5. The method of claim 4, wherein the at least one additional agent is a therapeutic agent.

6. The method of claim 5, wherein the therapeutic agent is a cholinesterase inhibitor.

7. The method of claim 4, wherein the at least one additional agent is administered in the same dosage form as the apilimod.

8. The method of claim 4, wherein the at least one additional agent is administered in a different dosage form from the apilimod.

9. The method of claim 7, wherein the dosage form is an oral dosage form.

10. The method of claim 1, wherein the dementia is Alzheimer's disease.

11. The method of claim 10, wherein the method is effective to alleviate at least one symptom of Alzheimer's disease in the patient, or effective to slow the progression of Alzheimer's disease in the patient.

12. The method of claim 1, wherein the apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine).

13. A method for inhibiting the processing of amyloid precursor protein (APP) into abeta (Ab) peptides in a cell, the method comprising contacting the cell with an amount of apilimod effective to inhibit APP processing into Ab peptides.

14. The method of claim 13, wherein the cell is in vitro or in vivo.

15. The method of claim 14, wherein the cell is part of a tissue.

16. The method of claim 15, wherein the tissue is neural tissue.

17. The method of claim 13, wherein the apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine).
